(19) United States
(12) Patent Application Publication
(10) Pub. No.: US 2012/0204875 A1
(43) Pub. Date: Aug. 16, 2012

(54) METHOD AND APPARATUS FOR MECHANICAL VENTILATION SYSTEM WITH DATA DISPLAY

(75) Inventors: Nathaniel David Brazy, Madison, WI (US); Timothy Patrick McCormick, Fitchburg, WI (US)

(73) Assignee: General Electric Company

(21) Appl. No.: 13/027,956

(22) Filed: Feb. 15, 2011

(51) Int. Cl. A61M 16/00

(52) U.S. Cl. 128/204.22; 128/204.18

(57) ABSTRACT

A mechanical ventilation system includes at least one processor in communication with a display device and at least one sensor configured to measure an aspect of the air carried by the ventilation system. The at least one processor is configured to receive and process data received from the at least one sensor. The processor is configured to generate and display on the display device a first graph of a measured aspect of the air corresponding to a first time period and a second graph of a measured aspect of the air corresponding to a second time period subsequent to the first time period. The at least one processor is configured to display the second graph superimposed over the first graph.

### BACKGROUND OF THE INVENTION

The present invention relates generally to the field of mechanical ventilation. The present invention relates specifically to the display of data related to mechanical ventilation.

A ventilator is a machine used during some medical treatments and procedures that assist or replace the spontaneous breathing of a patient. In brief, a mechanical ventilator system mechanically moves air into and out of the lungs of a patient. A ventilator may be used to provide breathing air to a patient who is unable to breathe on their own or is experiencing difficulty breathing, and in this manner, mechanical ventilation helps to maintain life of the patient who is having difficulty breathing. One type of mechanical ventilation is negative pressure ventilation (e.g., an iron lung) that generates negative pressure in a chamber surrounding the chest of a patient, and the negative pressure causes the chest to expand, drawing air into the lungs through the nose and mouth. Positive pressure ventilation is another type of ventilation in which pressurized air is used to deliver air into the lungs of the patient. Mechanical ventilation can be used to assist breathing during a number of medical conditions including acute lung injury, apnea, chronic obstructive pulmonary disease, respiratory acidosis, hypoxemia, hypotension, and certain neurological diseases such as muscular dystrophy and amyotrophic lateral sclerosis. Mechanical ventilation may also be used to assist breathing of newborns in neonatal intensive care. Further, mechanical ventilation may also be used to supply anesthetic agent to a patient undergoing certain medical procedures such as surgery.

### BRIEF DESCRIPTION OF THE INVENTION

One embodiment of the invention relates to a mechanical ventilation system including a pneumatic circuit configured to carry breathing air to a patient and to carry exhaled air from a patient and a display device. The mechanical ventilation system also includes at least one sensor associated with the pneumatic circuit that is configured to measure an aspect of the air carried by the pneumatic circuit and at least one processor in communication with the sensor and the display device. The at least one processor is configured to receive and process data received from the at least one sensor to generate and display on the display device a first graph of the measured aspect of the air corresponding to a first time period, and a second graph of the measured aspect of the air corresponding to a second time period subsequent to the first time period. The at least one processor is configured to display the second graph superimposed over the first graph.

Another embodiment of the invention relates to a control and display device configured for use in conjunction with a mechanical ventilation system that includes a sensor configured to measure a characteristic of the air carried by the ventilation system. The control and display device includes a display screen and a least one processor in communication with the display screen and the sensor. The at least one processor configured to receive and process data from the sensor to generate and display via the display screen a current waveform of the data received from the sensor corresponding to a most recent breath cycle of a patient and at least one prior waveform of the data received from the sensor corresponding to a prior breath cycle of the patient. The current waveform is displayed superimposed over the at least one prior waveform on a single set of axes.

Another embodiment of the invention relates to a method for controlling operation of a mechanical ventilation system to carry breathing air to a patient and to carry exhaled air from a patient. The method includes receiving a set of data representative of a characteristic of the air carried by the ventilation system and displaying on a display device a first waveform for a first breath cycle generated from the set of data. The method also includes overlaying a display of a second waveform for a subsequent breath cycle over the display of the first waveform, and the second waveform is generated from the set of data.

Alternative exemplary embodiments relate to other features and combinations of features as may be generally recited in the claims.

### BRIEF DESCRIPTION OF THE DRAWING

This application will become more fully understood from the following detailed description, taken in conjunction with the accompanying figures, wherein like reference numerals refer to like elements in which:

FIG. 1 is a diagram of a mechanical ventilator according to an exemplary embodiment;

FIG. 2 is a graph showing three ventilator waveforms displayed by a display device associated with a mechanical ventilator according to an exemplary embodiment;

FIG. 3 is a graph showing a ventilator waveform for a breath cycle displayed superimposed over waveforms of three prior breath cycles;

FIG. 4 is a graph showing a ventilator waveform for a breath cycle displayed superimposed over waveforms for a number of prior breath cycles;

FIG. 5 is a flow diagram showing the operation of a ventilator control system controlling a mechanical ventilation system according to an exemplary embodiment;

FIG. 6 is a flow diagram showing the operation of a ventilator control system controlling a mechanical ventilation system according to another exemplary embodiment;

FIG. 7 is a graph showing a ventilator pressure waveform displayed superimposed over a number of prior waveforms, according to an exemplary embodiment;

FIG. 8 is a graph showing a ventilator flow waveform displayed superimposed over a number of prior waveforms, according to an exemplary embodiment;

FIG. 9 is a graph showing a ventilator pressure waveform displayed superimposed over a number of prior waveforms, according to another exemplary embodiment;

FIG. 10 is a graph showing a ventilator pressure waveform displayed superimposed over a number of prior waveforms, according to another exemplary embodiment;

FIG. 11 is a graph showing a ventilator pressure waveform displayed superimposed over a number of prior waveforms, according to another exemplary embodiment; and

FIG. 12 is a graph showing a ventilator pressure waveform displayed superimposed over a prior waveform, according to another exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

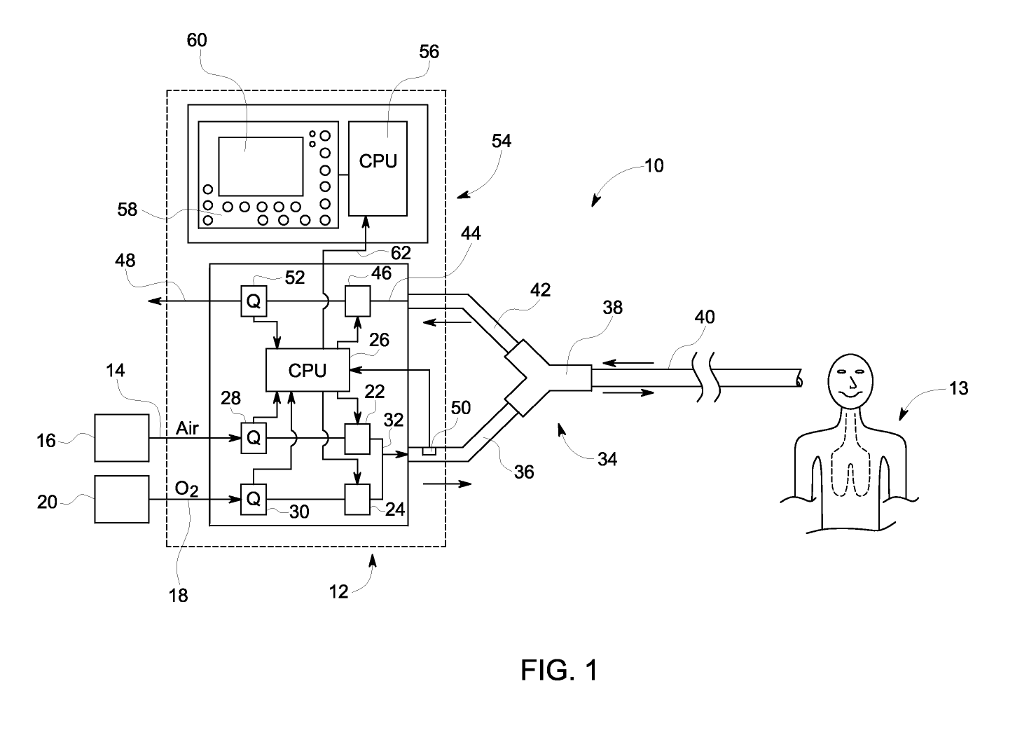

Referring to FIG. 1, a diagram of a mechanical ventilation system 10 is shown according to an exemplary embodiment. Generally, ventilation system 10 includes a ventilator 12, a breathing circuit 34 and a display and control unit 54 and is configured to deliver breathing air to a patient 13. Generally, ventilation system 10 includes a pneumatic circuit that carries breathing and exhaled air within ventilation system 10 and includes the various conduits of ventilator 12 and breathing circuit 34. An air conduit 14 supplies air to ventilator 12 from an air source 16, such as a container of pressurized air or a hospital air supply manifold. In one embodiment, an oxygen conduit 18 supplies oxygen to ventilator 12 from an oxygen source 20; for example, a container of compressed oxygen. The flow of air from air source 16 into ventilator 12 is controlled by valve 22, and the flow of oxygen from oxygen source 20 into ventilator 12 is controlled by valve 24.

Ventilator 12 includes a conduit 32 that receives the air and oxygen passing through valves 22 and 24, respectively. Conduit 32 is in communication with breathing circuit 34. The air and oxygen are mixed within conduit 32 and are then transmitted into inspiratory section 36 of breathing circuit 34. Breathing circuit 34 includes a Y-connector 38, and inspiratory section 36 is connected to a first arm of Y-connector 38. A second arm of Y-connector 38 is coupled to a patient segment 40 of breathing circuit 34. A distal end of patient segment 40 is coupled to the patient (e.g., via the nose, mouth, trachea, etc.). During inspiration (i.e., inhalation), breathing air is delivered through patient segment 40 of breathing circuit 34 and into the lungs of patient 13.

Breathing circuit 34 includes an expiration segment 42 coupled to a third arm of Y-connector 38. During expiration, expired or exhaled breathing air exits the lungs of patient 13 and is received into patient segment 40 of breathing circuit 34. The expired breathing air is communicated or transmitted through patient segment 40 and through Y-connector 38 and into expiration segment 42. Expiration segment 42 of breathing circuit 34 is coupled to ventilator 12, such that the expired air is received by ventilator 12 and communicated out of breathing circuit 34. As shown in FIG. 1, expiration segment 42 is in communication with a conduit 44 of ventilator 12. Ventilator 12 includes a valve 46 that controls flow of expired air into ventilator 12. With valve 46 in the open position, expired air flows from expiration segment 42 and into ventilator 12 for discharge from ventilator 12 through a discharge conduit 48.

While not specifically shown, ventilation system 10 may be equipped with various additional devices or systems as required for use in a particular situation, medical procedure, etc. In one embodiment, a nebulizer (not shown) can be positioned between the ventilator 12 and the inspiratory section 36 to introduce a medical drug (e.g., an anesthetic agent) to the breathing air of the patient as desired by the clinician. In other embodiments, breathing circuit 34 may include various components such as a humidifier to humidify the breathing air, a heater to heat the breathing air, or a water/vapor trap to remove excess moisture from the desired section of ventilation system 10.

Ventilation system 10 may include a variety of sensors to measure or read various aspects or characteristics (e.g., flow rate, pressure, volume, oxygen concentration, carbon dioxide concentration, etc.) of air within various sections of ventilator 12 or breathing circuit 34. As shown in FIG. 1, the air intake of ventilator 12 includes a sensor 28, and the oxygen intake of ventilator 12 includes a sensor 30. In one embodiment, sensors 28 and 30 are flow sensors configured to measure the rate of flow of air and oxygen into ventilator 12.

Ventilation system 10 also includes one or more sensors 50 located on the inspiratory section 36 of breathing circuit 34, and sensor 50 is configured to measure one or more aspects of the breathing air being delivered to patient 13. In various embodiments, sensor 50 may be a flow sensor configured to detect the inspiratory flow rate, a volume sensor configured to detect the volume of inspired air, or a pressure sensor configured to detect air pressure within breathing circuit 34 during inspiration. In addition, sensor 50 may be a sensor configured to measure the oxygen and/or carbon dioxide content of the air within breathing circuit 34 during inspiration. Ventilation system 10 may include a single sensor 50 or multiple sensors 50 to measure one or more of the characteristics of air discussed above.

Ventilation system 10 also includes one or more sensors 52 positioned to measure one or more aspects or characteristics of air being expired or exhaled from patient 13. In various embodiments, sensor 52 may be a flow sensor configured to detect the expiratory flow rate, a volume sensor configured to detect the volume of expired air, or a pressure sensor configured to detect air pressure within breathing circuit 34 during expiration. In other embodiments, sensor 52 may be a sensor configured to measure the oxygen and/or carbon dioxide content of the air within breathing circuit 34 during expiration. Ventilation system may include a single sensor 52 or multiple sensors 52 to measure one or more of the characteristics of expired air discussed above. It should be understood that while particular sensors are shown in the exemplary embodiment of FIG. 1, various sensors may be located at any suitable position within ventilator 12 or within breathing circuit 34 to provide the measurements discussed above or to provide the data for any of the one or more graphic displays discussed below.

Ventilation system 10 includes a control system configured to receive and process data received from the various sensors, user inputs, and any other desired data source (e.g., patient monitoring devices, such as ECG, EEG, pulse oximeters, etc., imaging data, hospital records, etc.) and to control various functionalities of ventilation system 10 as discussed herein. In the embodiment shown in FIG. 1, the control system of ventilation system 10 includes an electronic control circuit, shown as processor 26, located within ventilator 12. As shown, processor 26 is in communication with sensors 28, 30, 50 and 52 such that processor 26 receives data generated by the sensors.

In addition, in the embodiment shown in FIG. 1, the control system of ventilation system 10 includes a control and display device, shown as display unit 54. Display unit 54 includes an electronic control circuit, shown as processor 56, a user input device, shown as user interface 58, and a display device, shown as display screen 60. In this embodiment, processor 56 communicates with processor 26 via a communication link 62 (e.g., a data bus, a hard wired link, a wireless link, etc.). The control system of ventilation system 10 may be configured or equipped with one or more storage devices (e.g., memory, volatile memory, non-volatile memory, etc.) that is in communication with processors 26 and 56. Processors 26 and/or 56 may be configured to store, retrieve and delete data received from the various sensors, user inputs, and any other desired data source utilizing the one or more storage device as necessary to provide the display functionalities discussed herein. It should be understood that while the embodiment shown in FIG. 1 depicts a control system that includes processor 26 associated with ventilator 12 and a processor 56 associated with the display unit 54, a single processing circuit could be used, or more than two processing circuits may be used to provide the functionality discussed herein.

In the exemplary embodiment shown, the user (e.g., the clinician, doctor, nurse, etc.) may select various control parameters by interacting with user interface 58, and the control parameters are communicated to processor 26 or processor 56 to control the corresponding aspect of ventilation system 10 in accordance with the selected control parameter. In one embodiment, processor 26 is configured to control the operation of valves 22 and 24 to control flow of air and oxygen into ventilator 12 and to control operation of valve 46 to control flow of air out of breathing circuit 34 to ensure that the appropriate or desired breathing action is supplied by ventilator 12.

Display screen 60 of display unit 54 provides a visual display of various information associated with ventilation system 10. In various embodiments, information shown on display screen 60 may be viewed by the user to review the performance of ventilation system 10. Ventilator system 10 is an exemplary diagram of a ventilation system that may employ the display functionalities discussed herein. In one embodiment, ventilator system 10 may be an Engstrom Carestation type ventilation system available from GE Healthcare.

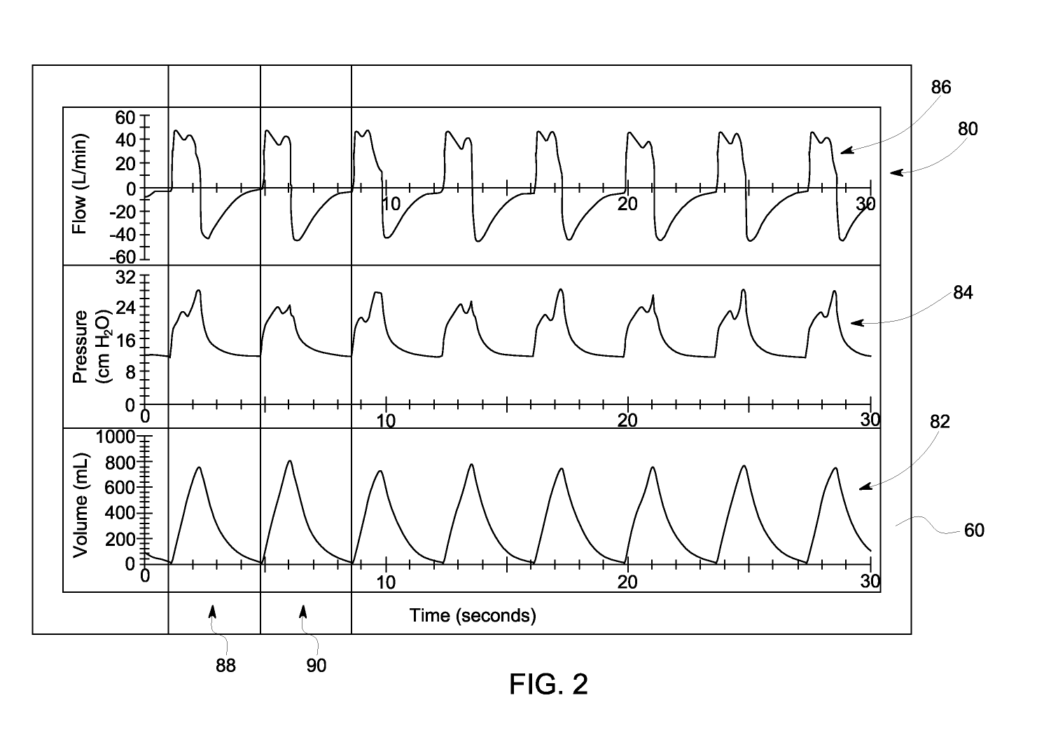

Referring to FIG. 2, a graphical display 80 is shown according to an exemplary embodiment. Graphical display 80 is an exemplary graphical display of data that may be displayed to the clinician/user via display screen 60 generated from data received from sensors 28, 30, 50 and 52. Graphical display 80 includes three sequential ventilator waveform graphs, a volume waveform 82, a pressure waveform 84, and a flow rate waveform 86. As shown, volume waveform 82 is a plot of air volume plotted versus time, pressure waveform 84 is a plot of pressure (e.g., air way pressure) versus time, and flow rate waveform is a plot of air flow rate versus time. Processor 26 and/or processor 56 is configured to receive and process data from sensors 28, 30, 50 and 52 or other suitable sensor and to generate and display waveforms 82, 84 and 86.

Because breathing is a cyclic process, waveforms 82, 84 and 86 are periodic having a cycle that generally repeats for each breath as shown in FIG. 2. Breath cycles may be defined as the cyclical segment of the waveform that occurs between sequential events during patient breath. By way of example, graphic display 80 shows eight individual breath cycles. In FIG. 2, the first displayed breath cycle is labeled 88 and the second displayed breath cycle is labeled 90. Breath cycles 88 and 90 start with the beginning of inhalation for the particular cycle and end at the beginning of inhalation of the next subsequent breath cycle.

Waveforms 82, 84 and 86 each display data representative of a measured aspect of air within ventilation system 10 plotted against time. Waveforms 82, 84 and 86 display each breath cycle in series or sequentially relative to the preceding and subsequent breath cycles such that each subsequent breath cycle is located at a new section of the time axis (i.e., the x-axis in FIG. 2). Thus, in this embodiment of graphical display 80, the waveforms for each subsequent breath cycle occur at different positions along the time axis of the graph and are not superimposed over the waveforms for earlier breath cycles.

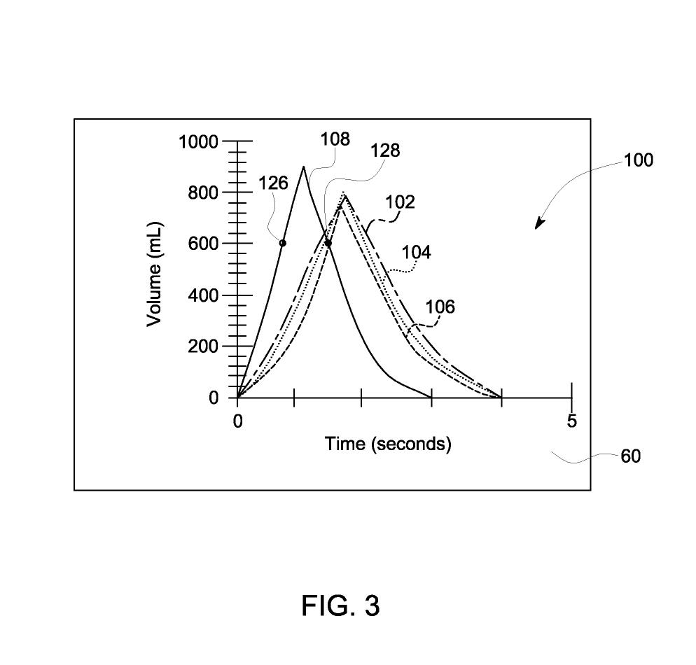

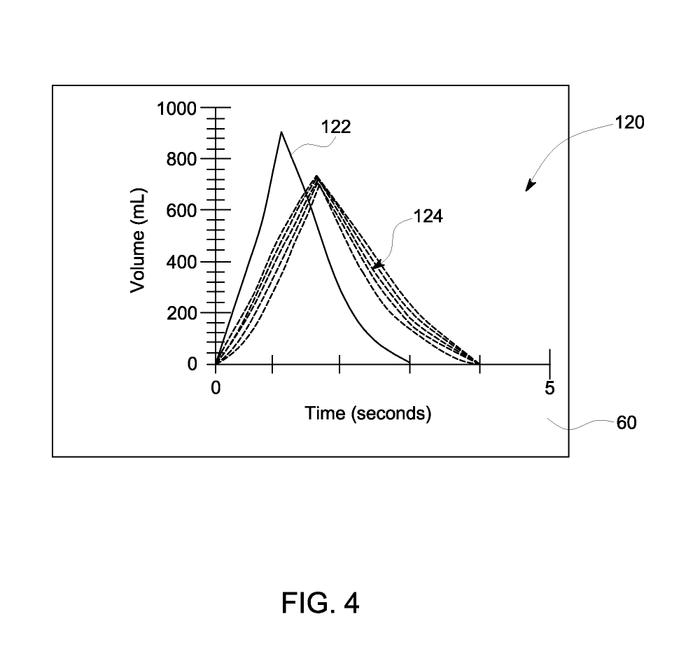

In other embodiments, ventilator system 10 may be configured to cause the display of various ventilator waveforms in other configurations that may be useful to the clinician or user of ventilator system 10. Referring generally to FIG. 3 and FIG. 4, the control system (e.g., processor 26 and/or processor 56) of ventilator system 10 is configured to cause the display of one or more graphs of ventilator data for one time period (e.g., a current breath cycle) overlaid or superimposed over one or more graphs of ventilator data for other, different time periods (e.g., one or more preceding breath cycles). The control system of ventilator system 10 is configured to receive and process the data from one or more of the sensors of ventilator system 10 to generate the superimposed waveform graphs discussed herein. Reviewing the display of the overlaid or superimposed periodic waveforms allows the operator of ventilation system 10 to more easily identify certain changes or shifts in the periodic waveforms as compared to reviewing sequential waveform plots such as that shown in FIG. 2.

Referring to FIG. 3, a graphical display 100 shows an overlay graph of ventilator waveform data that may be displayed via display screen 60 according to an exemplary embodiment. Graphical display 100 includes a first graph, shown as waveform 102, a second graph, shown as waveform 104, a third graph, shown as waveform 106, and a fourth graph, shown as waveform 108. Each waveform of graphical display 100 is a plot of a measured aspect or characteristic of air within ventilation system 10 (in this example: volume) for a particular time period plotted versus time following a trigger point. As shown in FIG. 3, waveform 108 represents data for the current or most recent breath cycle, and waveforms 102, 104, and 106 represent data for old or non-current breath cycles and are representative form a non-current ventilator data set 110. Each waveform 102, 104, 106 and 108 are plotted or superimposed over the waveforms for earlier breath cycles. In this embodiment, the control system maintains the display of a set number of previous or old breath cycles after the current breath cycle has been displayed.

In one embodiment, the control system (e.g., one or more electronic control circuit, processor, etc.) of ventilation system 10 is configured to display the waveform for the current breath cycle superimposed over a set number of non-current waveforms (e.g., N number of non-current waveforms). Thus, in an embodiment, the control system maintains the display of a set number of non-current breath cycles after the current breath cycle has been displayed. Referring to the display shown in FIG. 3, waveform 102 is the plot of data of the oldest or least recent breath cycle, waveform 104 is a plot of data for the breath cycle following the breath cycle of waveform 102, waveform 106 is a plot of data for the breath cycle following the breath cycle of waveform 104, and waveform 108 is a plot of data for the most recent or current breath cycle.

Referring to the display of FIG. 3, the control system is configured to display the most recent breath cycle and the waveforms for the three preceding breath cycles. As each new waveform is generated for each new breath cycle, a new current waveform 108 is displayed, the previous waveform 108 is moved to non-current data set 110 and becomes the new waveform 106. Previous waveform 106 becomes new waveform 104, previous waveform 104 becomes new waveform 102, and previous waveform 102 falls out of the non-current dataset and is no longer displayed as part of graphical display 100. Further, in contrast to the display shown in FIG. 2, graphical display 100 is a non-sequential or non-series plot (e.g., a plot in which each subsequent breath cycle does not occur at a different position along the time axis) of each subsequent breath cycle. It should be understood that while FIG. 3 shows only four total waveforms displayed (i.e., one current waveform and three non-current waveforms) any number (e.g., 1, 2, 4, 5, 6, 7, 8, 9, 10, etc.) of non-current waveforms may be displayed as desired by a user. In one embodiment, the user may select or control how many non-current waveforms are displayed via user interface 58.

This version of the superimposed waveform display may assist the user/clinician reviewing the displayed waveforms to identify a pattern or trend that is occurring slowly over a number of breath cycles. For example, as shown in FIG. 3, by superimposing current waveform 108 over non-current waveforms 102, 104 and 106, the user may easily identify that the volume of air inhaled in the current breath has increased sharply and that the duration of the current breath cycle has decreased which may indicate patient distress.

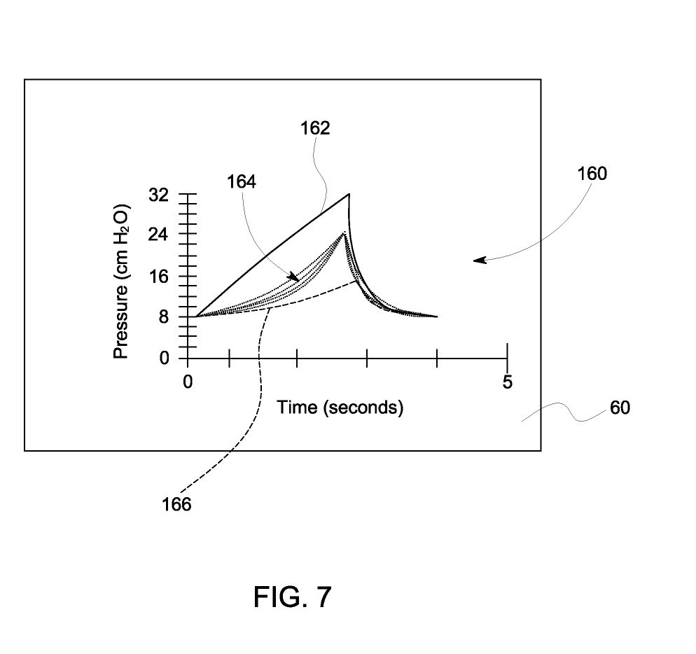

As another example, the user may identify gradual upward or downward shifts in the waveforms that occur over a number of breath cycles. Such trends may be identified more easily using an overlay plot as shown in FIG. 3 as compared to using a sequential breath cycle graph as shown in FIG. 2. Easily identifying such trends may allow the user to diagnose or identify a problem or abnormality with the patient or identify an issue with ventilator performance. For example, a gradual but steady increase in volume over a number of breath cycles may indicate over-inflation or increasing elasticity or compliance of the patient's lungs, and if such a condition is identified, the operation of ventilation system 10 may be adjusted (e.g., by changing a control parameter) as needed and/or other medical treatment can be supplied to the patient. As another example, the display of overlaid waveforms may allow the user to view and evaluate the effects of adjustments to various ventilator settings that may take several breath cycles to become identifiable via the waveform plots. Further, as discussed in more detail below, FIGS. 7-12 provide additional examples of several diagnoses or identifications that may be made by evaluating an overlay display of ventilator waveforms as discussed herein.

In one embodiment, the control system of ventilation system 10 may be configured to generate an animated display of waveforms 102, 104, 106 and 108. For example, following acquisition of the data corresponding to waveform 108, the control system may be configured to first display the entire waveform 102 at once, then to display the entire waveform 104 at once, then to display the entire waveform 106 at once, and then to display the entire current waveform 108 at once. In one embodiment, the display of each waveform is maintained during display of the other waveforms, and, in another embodiment, the display of each waveform is removed prior to the display of the next waveform. Thus, this display arrangement creates an animated display having the appearance of movement starting with the earliest waveform 102 and ending with the current waveform 108. The animated display configuration may help to highlight small, but steady changes in the waveforms that occur with each cycle.

According to another embodiment, FIG. 3 may represent a version of graphic display 80 in which the user may select the non-current waveforms to be display. The control system of ventilation system 10 may include an input device (e.g., user interface 58) that is configured to allow the user to identify and select one or more waveform to be displayed with the waveform for the current breath cycle. For example, in this embodiment, the user may select waveforms 102, 104 and 106 from a set of a number of prior waveforms for display along with the display of waveform 108 for the current breath cycle. In this embodiment, as each new breath cycle occurs, a new waveform 108 is displayed, but the user-selected waveforms 102, 104 and 106 do not change.

Thus, in this embodiment, the user may select one or more example or “snapshot” waveforms that the user wishes to compare against each new, current waveform. In the embodiment shown in FIG. 3, the user has selected three prior waveforms, waveforms 102, 104 and 106, for display along with each current waveform. However, in other embodiments, the user may select any number of prior waveforms to display along with the current waveform. In one such embodiment, the control system will allow the user to identify, label and save one or more waveform which then may be accessed for comparison, display or other analysis at a later date. In one such embodiment, the label and/or other identifying information (e.g., date of capture, patient information, ventilator settings, etc.) for the prior, “snapshot” waveforms may be displayed via display screen 60. Such identifying information may also be displayed for each new current waveform.

In various embodiments, the control system may be configured to display the waveforms 102, 104, 106 and 108 in a manner that allows the user to conveniently distinguish between each of the waveforms. In one embodiment, different colors and/or line intensities may be used to display each of the waveforms. In one embodiment, the intensity or brightness of the display of each waveform may be a function of the age of the waveform. For example, the intensity or brightness of the display of each waveform may decrease as the age of the waveform increases (e.g., the oldest data is the least bright and the current waveform is most bright). In another embodiment, as shown in FIG. 3, a different line style or weight can be used to distinguish between the waveforms from different breath cycles.

The display of user selected non-current waveforms may allow the user to compare one or more prior waveforms that are associated with a certain set of ventilator settings with the current waveform. In one such embodiment, if the current waveform is generated using the same ventilator settings as the prior waveforms, the user may evaluate or determine any source of deviation between the waveforms. In another embodiment, the current waveform may be generated using a different set of ventilator settings, allowing the user to evaluate the effects of different ventilator settings on patient respiration using ventilation system 10.

Referring to FIG. 4, a graphical display 120 shows an overlay graph of ventilator waveform data that may be displayed via display screen 60 according to another exemplary embodiment. In this embodiment, graphical display 120 includes a first graph, shown as waveform 122, that corresponds to the data for the current or most-recent breath cycle. Display 120 also includes a series of graphs, shown as non-current waveforms 124, that correspond to all the prior waveforms for each of the prior breath cycles. Thus, in this embodiment, the control system of ventilation system 10 may be configured to maintain or persist the display of all non-current waveforms for each of the prior breath cycles and to superimpose waveform 122 that corresponds to the current breath cycle over the display of the past non-current waveforms. Thus, graphical display 120 is useful in showing the amount of variation in waveform shape over an extended period of time, and it also is useful in showing any significant aberrations in the waveform shape that occurred during ventilator operation.

In one embodiment, current waveform 122 may be displayed in a different color, intensity or line style than non-current waveforms 124. As each new current waveform 122 is generated and displayed, the waveform 122 from the previous breath cycle is transferred to the group of non-current waveforms 124. In one embodiment, this transfer occurs by changing the color, intensity or line style of the waveform 122 from the previous breath cycle to match that of non-current waveforms 124.

In one embodiment, control system may be configured to allow the user to clear or erase the displays of non-current waveforms 124 via interaction with user interface 58. Further, control system may be configured to allow the user to select or identify the time period for which non-current waveforms 124 are displayed. For example, the user may select via user interface 58 a period of time and all waveforms for the set period of time, such as a set number of hours or days, are displayed as non-current waveforms 124. In one such embodiment, non-current waveforms 124 may be continuously displayed during a period when the clinician is not actively monitoring the displayed waveforms (e.g., overnight) such that the clinician can evaluate the consistency of ventilator operation and identify any aberrations that occurred during this period.

In various embodiments, the control system of ventilation system 10 may be configured to process the waveform data for each breath and to provide automated analysis and/or event warning based on this analysis. In one embodiment, the control system is configured to automatically analyze the non-current waveforms using proper statistical tools to identify a baseline waveform corresponding to normal patient breath or normal ventilator function. In this embodiment, the control system may be configured to then analyze or compare each current waveform to the baseline to detect any deviation above or below certain identified thresholds. The control system then may be configured to trigger an action (e.g., trigger an alarm, adjust ventilator operation settings, etc.) based on the detected deviation. In another embodiment, the control system is configured to provide a recommendation or suggested action (e.g., a suggested change to an operating parameter) to the user, and based upon this suggested action, the user may decide to take the suggested action.

In the embodiments discussed above, three general display configurations are discussed: the overlay graph or display of a set number of waveforms, the overlay graph of all of the waveforms for a particular time period, and the overlay graph of the waveform of the current breath with one or more “snapshot” waveforms. In one embodiment, the control system of ventilation system 10 may be capable of displaying all three display configurations and the user may select, via user interface 58, which display configuration to be used at a particular time.

In another embodiment, the control system of ventilation system 10 may be configured to shift data on the display in a manner to facilitate review and comparison of old and new data. In one such embodiment, the control system is configured to apply an upward and/or downward Y-axis shift to either the waveform for the current breath cycle or to the non-current waveforms. The Y-axis shift may allow the user to compare the shape of new and old waveforms without the old waveforms obstructing the view of the current waveform. In one embodiment, the user may be able to control the Y-axis shift via user interface 58.

As shown in FIG. 3 and FIG. 4, because breathing is cyclical, ventilator waveforms for each breath cycle may be displayed such that the beginning or trigger point for each waveform is positioned at the same point along the time axis (i.e., the x-axis in FIG. 3 and FIG. 4). Superimposing the waveforms in this manner helps to ensure that each superimposed waveform is aligned in a manner that facilitates comparison of the various waveforms by the user and allows the user to spot changes in waveform shape from cycle to cycle. Further, as noted above, the trigger point of the waveforms shown above is the start of inhalation such that in this embodiment each waveform is a plot of the measured aspect of the breath cycle starting at the beginning of inhalation for one breath cycle and ending immediately before inhalation of the next breath cycle.

However, in other embodiments other trigger points and/or other end points may be used. For example, the trigger point may be the start of expiration, the peak volume, flow rate, etc. In addition, the control system may be configured to display overlaid waveforms corresponding to periods of time other than a single breath cycle. For example, each individual waveform may correspond to multiple breath cycles, and in these embodiments, the trigger point may be every other inhalation, every third inhalation, every fourth inhalation, etc.

In other embodiments, the overlaid waveforms may correspond to a period of time that is less than a full breath cycle. In such sub-breath cycle plots, the trigger point and the end point of the displayed waveforms may be selected to highlight or enhance clinically important segments of the waveform. For example, referring to the volume waveforms of FIG. 3, alternative trigger point 126 may be selected at approximately 75 percent of inhalation volume and alternative end point 128 may be selected to be 75 percent of exhalation volume. In this embodiment, each of the displayed waveforms are plots of ventilator data for the time periods between alternative trigger point 126 and alternative end point 128 for each breath cycle with alternative trigger point 126 for each cycle being plotted at the same point on the time axis. In this embodiment, displaying overlaid waveforms of the upper 25 percent of the volume plot for each breath cycle may highlight differences in this section of the waveform in manner that easier to detect as compared to an overlay plot of the entire breath cycle.

In various embodiments, the control system of ventilation system 10 may be configured to allow the user to select or define the trigger point and/or end point via user interface 58 for the particular overlay graph that the user wishes to view. In various embodiments, the trigger point and/or end point may be selected for particular purposes (e.g., to highlight a clinically important region of the waveform plot). For example, the user may select the start of inhalation, the start of expiration, the peak volume, or any other desired event during the breath cycle as the trigger point.

Whether trigger points and end points are user selected or preprogrammed, the control system of ventilation system 10 may be configured to automatically identify the trigger point and generate the appropriate waveform display. For example, in assistive ventilation (i.e., ventilation in which inhalation is triggered by the patient's attempt to breath) the beginning of inhalation may be identified via analysis of the received sensor data. In fully-supported breathing applications, inhalation is started by operation of ventilator 12 and the control signal that controls the start of inhalation may also be used to trigger the plot of the waveform.

While FIGS. 3 and 4 show volume waveform plots for each breath cycle, the control system of ventilation system 10 may be configured to display waveforms of any of the data that may be measured via one or more sensors 28, 30, 50 and 52. For example, overlaid waveforms may be flow rate waveforms, pressure waveforms, oxygen concentration waveforms, carbon dioxide concentration waveforms, etc. In one embodiment, the user may select which type of waveforms to display via interaction with user interface 58.

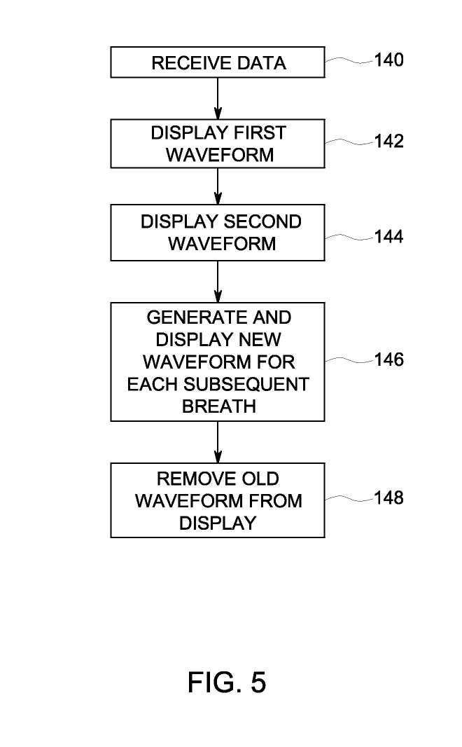

Referring to FIG. 5, a flow diagram showing the operation of a ventilator control system to control a mechanical ventilation system is shown according to an exemplary embodiment. At step 140 a set of data is received from a source, such as sensors 28, 30, 50 and 52, that represents a measured characteristic of air carried by the ventilation system. At step 142 a first waveform corresponding to a first time period (e.g., a first breath cycle) is generated from the data set and is displayed on a display device associated with the mechanical ventilation system. At step 144, a second waveform is generated from the data set corresponding to a subsequent breath cycle, and the second waveform is displayed overlaid over the display of the first waveform. At step 146, a new waveform is generated from the data set corresponding to each new, subsequent breath cycle, and each new waveform is displayed overlaying the displayed waveform for at least the immediately proceeding breath cycle. In one embodiment all of the prior or non-current waveforms for a period of time may be displayed as shown in FIG. 4. In another embodiment, at step 148, the oldest non-current waveform may be removed from the display after it has been displayed for a predetermined number of breath cycles such that only a set number of prior or non-current waveforms may be displayed as shown in FIG. 3.

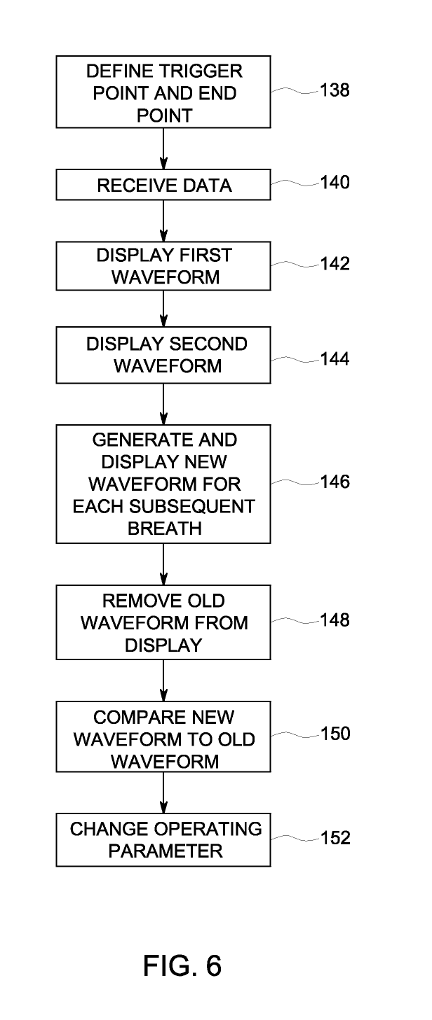

Referring to FIG. 6 a flow diagram showing the operation of a ventilator control system to control a mechanical ventilation system is shown according to another exemplary embodiment. This embodiment is similar to the method shown in FIG. 5. However, in this embodiment, at step 138 a trigger point that identifies the beginning of each waveform plot and/or an end point that identifies the end of each waveform plot are defined based on an input received from a user. At step 150, a subsequent waveform (e.g., the current waveform) is compared to one or more of the non-current waveforms to identify an abnormality in the breathing of the patient or in the operation of the ventilation system. In one such embodiment, the current waveform may be compared to one or more of the non-current waveforms to identify whether the elasticity of the patient's lungs is decreasing. At step 152, an operating parameter of the ventilation system may be changed or adjusted based on the comparison performed at step 150.

In one embodiment, the control system of ventilation system 10 is an electronic control system programmed to perform methods shown and discussed above. In particular, the control system may include non-transitory programmed instructions for performing each of the steps shown in FIGS. 5 and 6 or to generate the displays as shown and described above regarding FIGS. 2, 3 and 4. In another embodiment, computer readable media is provided to control operation of a mechanical ventilation system, and the computer readable media includes programmed non-transitory instruction for performing each of the steps shown in FIGS. 5 and 6 or to generate the displays as shown and described above regarding FIGS. 2, 3 and 4.

Referring to FIGS. 7-12, several overlay waveform displays are shown according to various exemplary embodiments. Referring to FIG. 7, a graphical display 160 shows an overlay graph of ventilator pressure waveform data that may be displayed via display screen 60. In this embodiment, graphical display 160 is a display of waveform data from a ventilator operating in volume control mode (i.e., a mode in which the ventilator ensures a set volume of air is delivered to the patient with each breath).

In this embodiment, graphical display 160 includes a first graph, shown as waveform 162, that corresponds to the data for the current or most-recent breath cycle. Display 160 also includes a series of graphs, shown as non-current waveforms 164, that correspond to the data of all the prior waveforms for each of the prior breath cycles during a set time period. The upward angled portion of each waveform of display 160 corresponds to the inhalation or inspiratory phase of the breath cycle, and in this mode of ventilator operation, the slope of the upward angled portion and the peak of the waveform are inversely related to the compliance of the patient's lungs (e.g., the ability of the lungs to stretch during a change in pressure). Thus, a lower slope of the upward angled portion of the waveform and a lower peak of the waveform corresponds to a higher lung compliance, and a higher slope of the upward angled portion of the waveform and a higher peak of the waveform corresponds to a lower lung compliance. In addition, decreasing lung compliance may indicate that a patient's breathing condition or effectiveness is declining.

As shown in graphical display 160, the slope of the upward section and the peak of current waveform 162 has increased relative to prior waveforms 164 indicating a decrease in lung compliance which indicates that the patient's condition is worsening. Graphical display 160 also shows an alternative current waveform 166 that has a slope and peak that is less than prior waveforms 164 indicating an increase in lung compliance which indicates that the patient's condition is improving. Thus, superimposing a waveform 162 over prior waveforms 164 may help the user to identify changes in the waveform shape and, in particular, changes in slope of the waveform, more easily than if each waveform were viewed in series. When the clinician identifies an increase or decrease in lung compliance by viewing display 160, the clinician may take appropriate action such as to adjust an operating parameter of the ventilator or perform an appropriate medical intervention or procedure.

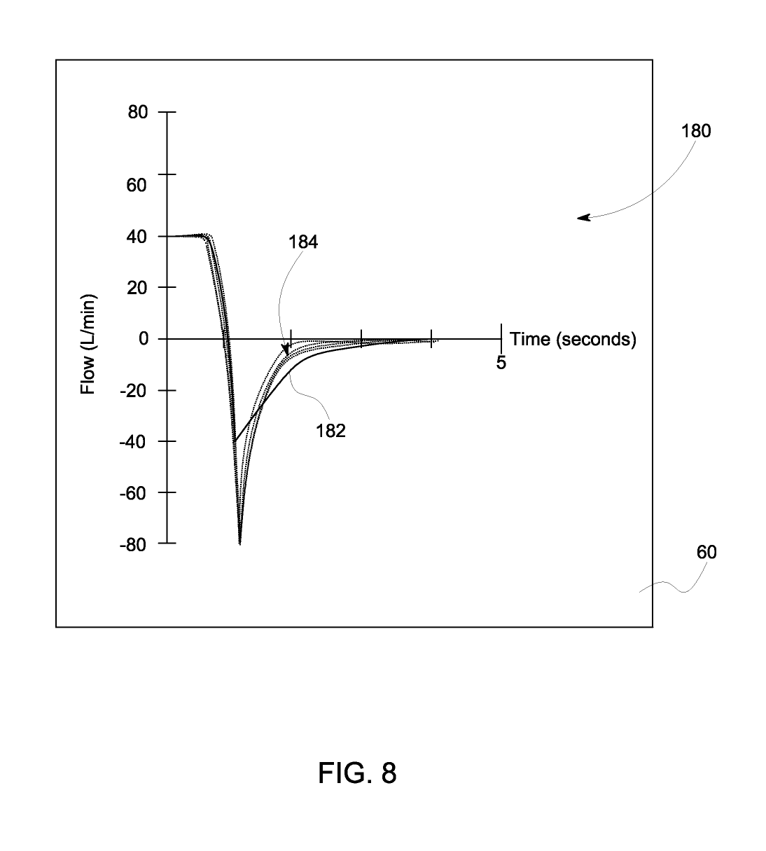

Referring to FIG. 8, a graphical display 180 shows an overlay graph of ventilator air flow waveform data that may be displayed via display screen 60. Similar to display 160 shown in FIG. 7, graphical display 180 of FIG. 8 is a display of waveform data from a ventilator operating in volume control mode (i.e., a mode in which the ventilator ensures a set volume of air is delivered to the patient with each breath).

In this embodiment, graphical display 180 includes a first graph, shown as waveform 182, that corresponds to the data for the current or most-recent breath cycle. Display 180 also includes a series of graphs, shown as non-current waveforms 184, that correspond to data from all the prior waveforms for each of the prior breath cycles during a set time period. Graphical display 180 is an example of an overlay display of waveform data from a portion of each breath cycle. In this embodiment, graphical display 180 generally shows the flow rate of the expiratory portion of the breath cycle. Thus, in this embodiment the trigger point for waveform display is the start of expiration and the end point of waveform display is the point where expiratory flow returns to zero. By utilizing these trigger and end points, display 180 specifically displays an overlay of clinically significant portions of the flow waveform in this embodiment.

A plot of the flow waveform data during the expiratory portion of the breathing cycle provides information regarding resistance within the breathing circuit and within the patient's lungs and airway. Referring to FIG. 8, during expiration, lower expiratory resistance is indicated by a lower (i.e., a more negative) peak of the flow waveform. Lower expiratory resistance is also indicated by the flow waveform taking a smaller amount of time to return close to zero (i.e., to approach the x-axis, to cross the x-axis, etc.). Lower resistance within the patient's airway and lungs is an indication of good patient health, and lower resistance within the ventilator indicates that the breathing circuit is clear of significant obstruction. Increasing resistance may indicate that the patient's lungs or airway are becoming obstructed (e.g., with mucus, fluid, etc.) which may indicate a decrease in the patient's health.

As shown in graphical display 180, the peak of current waveform 182 has become less negative indicating that maximum expiratory flow rate has decreased relative to prior waveforms 184, and the period of current waveform 182 (i.e., the time from start of expiration to the point where flow rate approaches zero) has increased indicating that it is taking longer for expiration to occur relative to prior waveforms 184. These changes provide an indication that resistance within the patient's lungs or airway or within the breathing circuit is increasing.

Superimposing current waveform 182 over prior waveforms 184 may help the user to identify changes in the waveform shape and, in particular, changes in slope of the waveform, more easily than if each waveform were viewed in series. When the clinician identifies an increase in resistance based on display 180, the clinician may take appropriate action to lower resistance. Such actions may include removing an obstructing substance from the patient's lungs or airway or may include removing an obstructing substance from the breathing circuit. In one exemplary embodiment, the obstructing substance may be removed from the breathing circuit by applying suction to the breathing circuit.

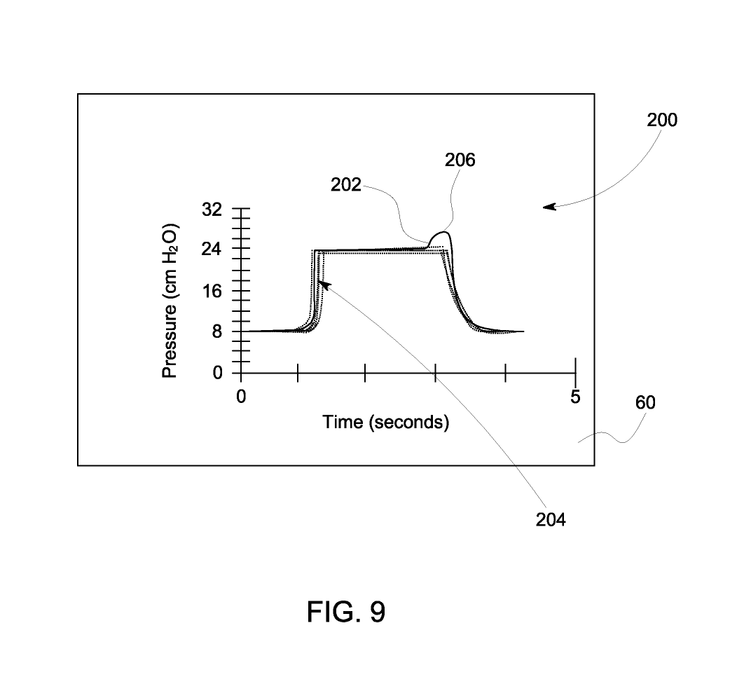

Referring to FIG. 9, a graphical display 200 shows an overlay graph of ventilator pressure waveform data that may be displayed via display screen 60. In this embodiment, graphical display 200 is a display of waveform data from a ventilator operating in pressure control mode (i.e., a mode in which the ventilator ensures a set pressure is delivered for a set period of time with each breath). In this embodiment, graphical display 200 generally shows the pressure waveform of the inspiratory portion of the breath cycle. Thus, in this embodiment the trigger point for waveform display 200 is the start of inspiration and the end point of waveform display is set a short time following the end of inspiration. By utilizing these trigger and end points, display 200 specifically displays an overlay of clinically significant portions of the pressure waveform in this embodiment.

In this embodiment, graphical display 200 includes a first graph, shown as waveform 202, that corresponds to the data for the current or most-recent breath cycle. Display 200 also includes a series of graphs, shown as non-current waveforms 204, that correspond to the data of all the prior waveforms for each of the prior breath cycles during a set time period. In certain applications, the ventilator may deliver breathing air to the patient independent of the patient's natural attempts to breath. In this situation, if the ventilator is not synchronized with the patient's natural attempts to breath, the patient's attempt to breath may act against the action of the ventilator leading to inefficiency in the delivery of breathing air by the ventilator.

Referring to FIG. 9, in pressure control mode the ideal pressure waveform should approximate a square waveform, similar to non-current waveforms 204. If the patient's attempt to breath is not synchronized with the ventilator, the patient may attempt to exhale while the ventilator is still supplying pressure to drive breathing air into the patient's lungs. As the patient's body attempts to exhale, a spike 206 in pressure may be visible in current pressure waveform 202 indicating that the patient's natural breathing cycle is not synchronized with the breathing cycle of the ventilator. When the clinician identifies the presence of spike 206 by viewing display 200, the clinician may take appropriate action such as adjusting the timing of ventilator breathing cycles to better synchronize with the patient's natural breathing cycle. Further, by displaying overlaid waveforms generated over a long period of time (e.g., over night, over one or more days, etc.), the clinician may detect that the frequency of unsynchronized breath attempts is changing (e.g., increasing or decreasing), and may alter ventilator operation accordingly.

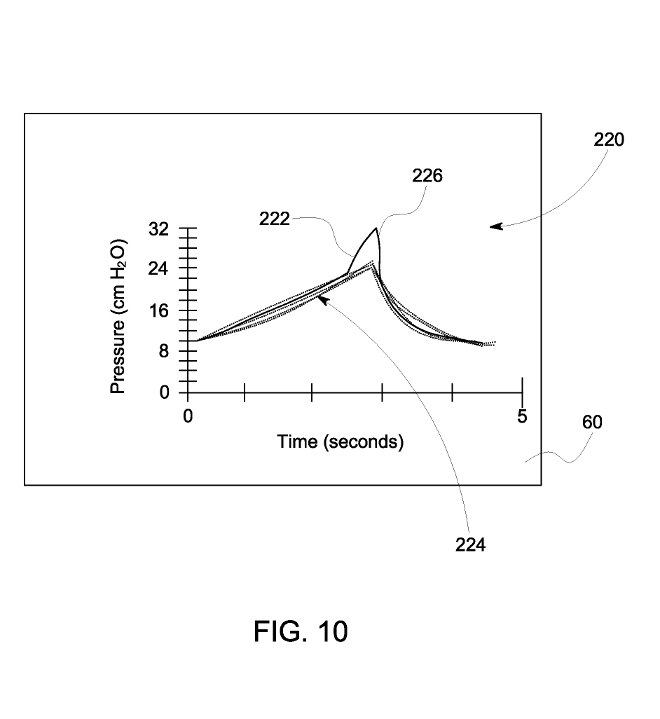

Referring to FIG. 10, a graphical display 220 shows an overlay graph of ventilator pressure waveform data that may be displayed via display screen 60. In this embodiment, graphical display 220 is a display of pressure waveform data from a ventilator operating in volume control mode (i.e., a mode in which the ventilator ensures a set volume of air is delivered to the patient with each breath). Graphical display 220 includes a first graph, shown as waveform 222, that corresponds to the data for the current or most-recent breath cycle. Display 220 also includes a series of graphs, shown as non-current waveforms 224, that correspond to the data of all the prior waveforms for each of the prior breath cycles during a set time period. Similar to the embodiment of FIG. 9, current pressure waveform 222 may show a spike 226 generated by the patient's attempt to breath indicating that the ventilator breathing cycle is not synchronized with the patient's attempts to breath.

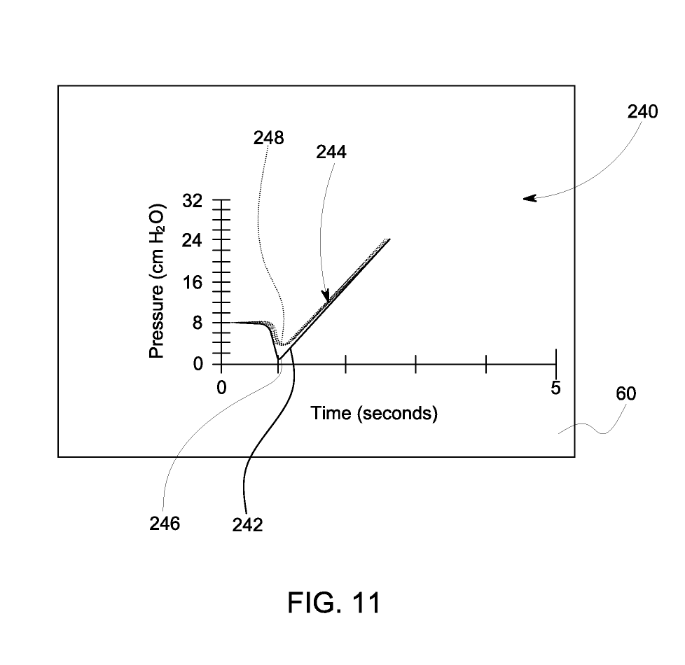

Referring to FIG. 11, a graphical display 240 shows an overlay graph of ventilator pressure waveform data that may be displayed via display screen 60. In this embodiment, graphical display 240 includes a first graph, shown as waveform 242, that corresponds to the data for the current or most-recent breath cycle. Display 240 also includes a series of graphs, shown as non-current waveforms 244, that correspond to data from all the prior waveforms for each of the prior breath cycles during a set time period. In this embodiment, graphical display 240 generally shows the pressure waveform of the inspiratory portion of the breath cycle. Thus, in this embodiment the trigger point for waveform display 240 is the start of inspiration and the end point of waveform display is near the peak of the waveform. By utilizing these trigger and end points, display 240 specifically displays an overlay of clinically significant portions of the flow waveform in this embodiment.

In certain applications, a patient that is breathing with the assistance of a ventilator may be capable of trying to breath on their own, and in some embodiments, this inspiratory effort by the patient may be detected the ventilator and may be used to start or trigger inspiration by the ventilator. As the patient attempts to inhale, the patient's lungs expand causing a slight drop in pressure within the breathing circuit. This momentary drop in pressure is visible as depression 246 in current waveform 242 and as depressions 248 in non-current waveforms 248. The shape and minimum point of depression 246 and depression 248 provide an indication of the strength of the inspiratory effort by the patient. In particular, the greater the depression (i.e., the closer the minimum point is to the x-axis) the stronger the inspiratory effort by the patient, and increasing inspiratory effort by the patient indicates that the patient's lungs and associated muscles are getting stronger and healthier. Thus, display 240 may depict trends in the size and shape of depressions 246 and 248 over a period of time, allowing the clinician to evaluate whether the patient's condition is static, improving or declining based on the changing size and shape of depressions 246 and 248.

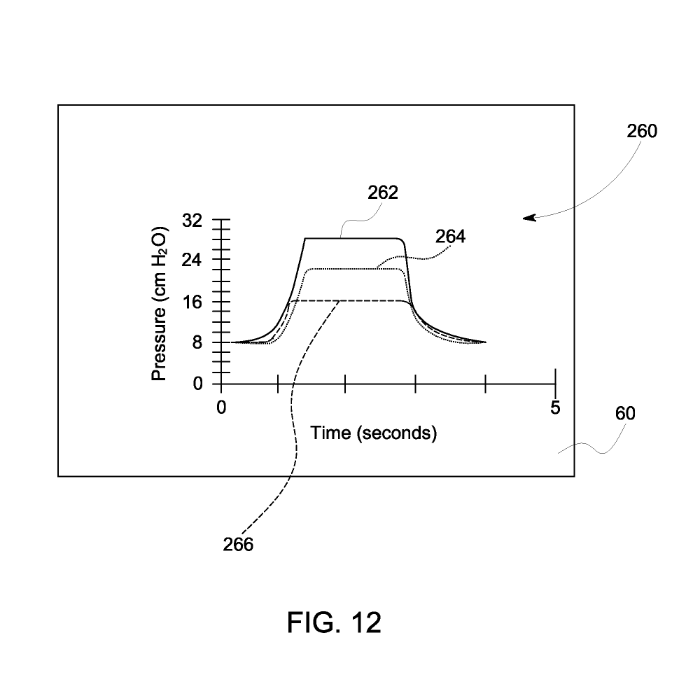

Referring to FIG. 12, a graphical display 260 shows an overlay graph of ventilator pressure waveform data that may be displayed via display screen 60. In this embodiment, graphical display 260 is a display of waveform data from a ventilator operating in pressure controlled/volume guarantee mode (i.e., a mode in which the ventilator ensures a set volume of air is delivered with each breath cycle while also ensuring that the pressure remains within predefined limits).

In this embodiment, graphical display 260 includes a first graph, shown as waveform 262, that corresponds to the data for the current or most-recent breath cycle. Display 260 also includes a graph (or series of graphs), shown as non-current waveform 264, that corresponds to the data of one or more prior waveforms for one or more prior breath cycles. Graphical display 260 also shows an alternative current waveform 266. In this embodiment, the maximum pressure of the waveform is inversely related to the compliance of the patients lungs because as the compliance of the patient's lungs decreases, a higher pressure is needed to supply a set volume of air to a patient within a fixed period of time. Accordingly, alternative current waveform 266 corresponds to more compliant lungs compared to waveforms 262 and 264, and current waveform 262 corresponds to less compliant lungs compared to waveforms 264 and 266. Further, as noted above, more compliant lungs are typically associated with better patient health or improving patient condition. When the clinician identifies an increase or decrease in lung compliance by viewing display 260, the clinician may take appropriate action such as to adjust an operating parameter of the ventilator and perform an additional medical intervention.

FIGS. 7-12 provide various examples of overlaid waveform displays that correspond to various patient conditions and various aspects of ventilator operation. The control system of ventilation system 10 may be configured to process the waveform data for each breath and to provide automated analysis, event warning, automated ventilator control and/or automated suggestions or recommendations to the user based on the analysis of the waveform data for any of the waveform types, patient conditions and ventilator operating conditions discussed above. In one embodiment, the control system may be configured to provide a suggestion or recommendation to the user regarding a change in a timing parameter of the ventilation system to better synchronize ventilator breathing with the patient's natural breathing attempts. In another embodiment, the control system may be configured to provide a suggestion to the user regarding whether to clear the breathing circuit based on a detected change in resistance. In various embodiments, the recommendation may be in the form of a icon or text displayed on the display screen or an auditory signal.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. Further modifications and alternative embodiments of various aspects of the invention will be apparent to those skilled in the art in view of this description. The construction and arrangements, shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments, without departing from the scope of the present invention.

What is claimed is:

1. A mechanical ventilation system comprising:
a pneumatic circuit configured to carry breathing air to a patient and to carry exhaled air from a patient;
a display device;
at least one sensor associated with the pneumatic circuit, the at least one sensor configured to measure an aspect of the air carried by the pneumatic circuit; and
at least one processor in communication with the at least one sensor and the display device, the at least one processor configured to receive and process data received from the at least one sensor to generate and display on the display device:
a first graph of the measured aspect of the air corresponding to a first time period; and
a second graph of the measured aspect of the air corresponding to a second time period subsequent to the first time period;
wherein the at least one processor is configured to display the second graph superimposed over the first graph.

2. The mechanical ventilation system of claim 1, wherein the first and second graphs are graphs of the measured aspect of the air versus time, and further wherein the first time period and the second time period both begin at a trigger point in each breathing cycle of the patient.

3. The mechanical ventilation system of claim 2, wherein the second time period is the most recent breath cycle and the first time period is the breath cycle immediately preceding the most recent breath cycle, wherein the trigger point is the beginning of inhalation of each breath cycle such that the displayed graphs depict the measured data for two consecutive breath cycles superimposed on a single set of axes, wherein the trigger points for each breath cycle are located at the same point along the time axis.

4. The mechanical ventilation system of claim 3, wherein the at least one processor is configured to generate and display on the display device an additional graph of the measured aspect of the air for each prior consecutive breath cycle, and further wherein the at least one processor is configured to display the second graph superimposed over the first graph and over at least one of the additional graphs.

5. The mechanical ventilation system of claim 4, wherein the at least one processor is configured to maintain the display of a set number of the additional graphs.

6. The mechanical ventilation system of claim 4, wherein the at least one processor is configured to maintain the display of all of the additional graphs displayed within a set time period.

7. The mechanical ventilation system of claim 3, wherein the at least one processor is configured to automatically detect the start of each breath cycle and to display each graph such that start of each breath cycle of each graph is located at the same point on the time axis of the graph.

8. The mechanical ventilation system of claim 2, further comprising a set of additional graphs each corresponding to a previous breath cycle, further comprising a user input device configured to allow the user of the mechanical ventilator system to select one or more of the first graph and the additional graphs to be displayed along with the second graph.

9. The mechanical ventilator system of claim 2, further comprising a user input device configured to allow the user of the mechanical ventilator system to select the trigger point that identifies the beginning of each displayed waveform.

10. The mechanical ventilator system of claim 1, wherein the at least one processor is configured to analyze the second graph to identify a deviation of the second graph from the first graph and to trigger an alarm if the deviation exceeds a threshold.

11. The mechanical ventilator system of claim 1, wherein the measured aspect of air carried by the breathing circuit is at least one of volume, pressure, flow rate, oxygen concentration, and carbon dioxide concentration.

12. A control and display device configured for use in conjunction with a mechanical ventilation system that includes a sensor configured to measure a characteristic of the air carried by the ventilation system, the control and display device comprising:
a display screen; and
a least one processor in communication with the display screen and the sensor, the at least one processor configured to receive and process data from the sensor to generate and display via the display screen:
a current waveform of the data received from the sensor corresponding to a most recent breath cycle of a patient; and
at least one prior waveform of the data received from the sensor corresponding to a prior breath cycle of the patient;
wherein the current waveform is displayed superimposed over the at least one prior waveform on a single set of axes.

13. The control and display device of claim 12 wherein the at least one processor is further configured to generate and display the current waveform superimposed over a plurality of prior waveforms to create an animated display of the waveforms.

14. A method for controlling operation of a mechanical ventilation system to carry breathing air to a patient and to carry exhaled air from the patient, the method comprising:
receiving a set of data representative of a characteristic of the air carried by the ventilation system;
displaying on a display device a first waveform for a first breath cycle generated from the set of data; and
overlaying a display of a second waveform for a subsequent breath cycle over the display of the first waveform, the second waveform generated from the set of data.

15. The method of claim 14 further comprising defining a trigger point that defines the beginning of each breath cycle based on an input received from a user.

16. The method of claim 14 further comprising comparing the second waveform to the first waveform to identify an abnormality in the patient's breathing or an abnormality in the operation of the mechanical ventilation system.

17. The method of claim 16 further comprising changing an operating parameter of the mechanical ventilation system based on the comparison of the second waveform to the first waveform.

18. The method of claim 16 wherein the second waveform is compared to the first waveform to identify information related to at least one of: compliance of the patient's lungs, resistance within the patient's airway, resistance within the ventilation system, synchronization between the patient's natural breathing cycle and the breathing cycle of the ventilator, and an inspiratory effort of the patient.

19. The method of claim 16 further comprising displaying a new waveform for each subsequent breath cycle, each new waveform overlaying the displayed waveform for at least the immediately preceding breath cycle.

20. The method of claim 19 further comprising removing a waveform from the display once it has remained on the display for a predetermined number of breath cycles.
